(19) United States
(12) Patent Application Publication
(10) Pub. No.: US 2024/0009152 A1
(43) Pub. Date: Jan. 11, 2024

(54) METHODS OF MANUFACTURE OF SURAMIN

(71) Applicant: Perfect Daylight Limited, Monaco (MC)

(72) Inventors: Matthew E. CALDER, Denver, CO (US); Elso DIFRANCO, Chesterfield, VA (US); Keith L. SPENCER, Lyons, CO (US)

(21) Appl. No.: 18/301,077

(22) Filed: Apr. 14, 2023

Related U.S. Application Data
(63) Continuation of application No. 18/102,477, filed on Jan. 27, 2023, now abandoned.
Application No. 18/102,477 is a continuation of application No. PCT/US21/43574, filed on Jul. 28, 2021.
(60) Provisional application No. 63/058,076, filed on Jul. 29, 2020.

(51) Int. Cl. A61K 31/167; B01J 23/44

(52) U.S. Cl. CPC A61K 31/167; B01J 23/44

(57) ABSTRACT

Pharmaceutical compositions comprising suramin and methods of preparing synthetic intermediates useful for the preparation of suramin are described herein.

### CROSS-REFERENCE

This application is a continuation of U.S. patent application Ser. No. 18/102,477, filed Jan. 27, 2023; which is a continuation of PCT Application No. PCT/US21/43574, filed Jul. 28, 2021; which claims the benefit of U.S. Provisional Application No. 63/058,076, filed Jul. 29, 2020; each of which are entirely incorporated herein by reference for all purposes.

### BACKGROUND

Suramin, a urea compound useful in the treatment of African sleeping sickness and river blindness, was developed by chemists at Bayer in the early 1900s.

In recent years, suramin has shown promise in the treatment of autism, but its potential utility has been limited by problems with existing methods of its manufacture. Formation of the urea bond is accomplished with phosgene, a highly toxic gas that synthetic chemists have largely replaced with more benign alternatives. Further, the existing synthetic methods do not provide suramin to a high degree of purity.

Because new potential therapeutic uses of suramin have been discovered, there is a need for a modern manufacturing method that can produce suramin in high yield and purity without the use of harsh reaction conditions and dangerous reagents.

### SUMMARY

Disclosed herein, in certain embodiments, pharmaceutical compositions and methods of preparing compounds. More particularly, the disclosure relates to pharmaceutical compositions comprising suramin and methods of preparing synthetic intermediates useful for the preparation of suramin.

In one aspect, the present disclosure provides a pharmaceutical composition comprising a substantially pure composition of a compound of Formula I:

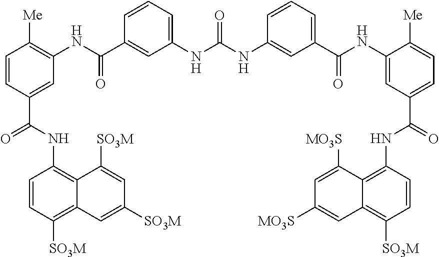

and a pharmaceutically acceptable excipient, wherein M is each independently H, Li, Na, or K. In some embodiments, M is each independently H, Na, or K. In some embodiments, M is each independently H, Li, or K. In some embodiments, M is each independently H, Na, or Li. In some embodiments, M is each independently H or Na. In some embodiments, M is each independently H or K. In some embodiments, M is each independently H or Li. In some embodiments, M is Na. In some embodiments, M is H. In some embodiments, M is Li. In some embodiments, M is K.

In some embodiments, the substantially pure composition of the compound of Formula I comprises, by weight or by mole, at least about 95% of the compound of Formula I. In some embodiments, the substantially pure composition of the compound of Formula I comprises, by weight or by mole, at least about 97% of the compound of Formula I. In some embodiments, the substantially pure composition of the compound of Formula I comprises, by weight or by mole, about 95% to about 99.9%, about 96% to about 99.9%, about 97% to about 99.9%, about 98% to about 99.9%, or about 99% to about 99.9% of the compound of Formula I. In some embodiments, the substantially pure composition of the compound of Formula I comprises, by weight or by mole, about 95% to about 99.99%, about 96% to about 99.99%, about 97% to about 99.99%, about 98% to about 99.99%, or about 99% to about 99.99% of the compound of Formula I.

In some embodiments, the substantially pure composition of the compound of Formula I comprises an impurity of Formula I-A

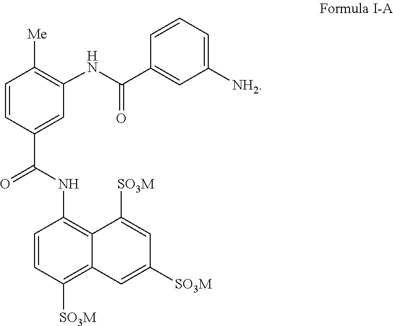

In some embodiments, the substantially pure composition of the compound of Formula I comprises, by weight or by mole, less than about 5% of the impurity of Formula I-A. In some embodiments, the substantially pure composition of the compound of Formula I comprises, by weight or by mole, less than about 3% of the impurity of Formula I-A. In some embodiments, the substantially pure composition of the compound of Formula I comprises, by weight or by mole, about 0.01% to about 10%, about 0.01% to about 9%, about 0.01% to about 8%, about 0.01% to about 7%, about 0.01% to about 6%, about 0.01% to about 5%, about 0.01% to about 4%, about to about 3%, about 0.01% to about 2%, about 0.01% to about 1%, or about 0.01% to about of the impurity of Formula I-A. In some embodiments, the substantially pure composition of the compound of Formula I comprises, by weight or by mole, about 0.005% to about 10%, 0.005% to about 9%, 0.005% to about 8%, 0.005% to about 7%, 0.005% to about 6%, 0.005% to about 5%, to about 4%, 0.005% to about 3%, 0.005% to about 2%, 0.005% to about 1%, or 0.005% to about 0.5% of the impurity of Formula I-A. In some embodiments, the substantially pure composition of the compound of Formula I comprises, by weight or by mole, about 0.001% to about 10%, about 0.001% to about 9%, about 0.001% to about 8%, about 0.001% to about 7%, about to about 6%, about 0.001% to about 5%, about 0.001% to about 4%, about 0.001% to about 3%, about 0.001% to about 2%, about 0.001% to about 1%, or about 0.001% to about 0.5% of the impurity of Formula I-A.

In another aspect, the present disclosure provides a method of treating an autism spectrum disorder in a subject in need thereof, wherein the method comprises administering to the subject a therapeutically effective amount of the pharmaceutical composition disclosed herein. In some embodiments, the pharmaceutical composition is administered to the subject intravenously, intranasally, subcutaneously, or parenterally. In some embodiments, the pharmaceutical composition is administered to the subject intravenously.

In another aspect, the present disclosure provides a method of treating fragile X-associated tremor/ataxia (FXTAS) in a subject in need thereof, wherein the method comprises administering to the subject a therapeutically effective amount of the pharmaceutical composition disclosed herein. In some embodiments, the pharmaceutical composition is administered to the subject intravenously, intranasally, subcutaneously, or parenterally.

In another aspect, the present disclosure provides a method of preparing a compound of Formula I-A

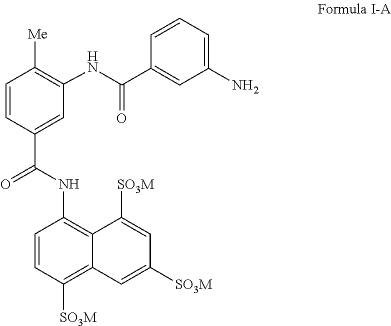

from a compound of Formula I-B

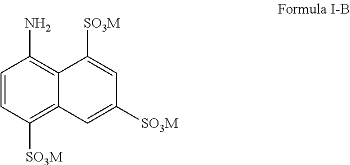

wherein M is each independently H, Li, Na, or K, and wherein the method provides the compound of Formula I-A in an overall yield of greater than about 80%. In some embodiments, M is each independently H, Na, or K. In some embodiments, M is each independently H, Li, or K. In some embodiments, M is each independently H, Na, or Li. In some embodiments, M is each independently H or Na. In some embodiments, M is each independently H or K. In some embodiments, M is each independently H or Li. In some embodiments, M is Na. In some embodiments, M is H. In some embodiments, M is Li. In some embodiments, M is K.

In some embodiments, the method provides the compound of Formula I-A in an overall yield of greater than about 90%. In some embodiments, the method provides the compound of Formula I-A in an overall yield of greater than about 81%, greater than about 82%, greater than about 83%, greater than about 84%, greater than about 85%, greater than about 86%, greater than about 87%, greater than about 88%, greater than about 89%, or greater than about 90%. In some embodiments, the method provides the compound of Formula I-A in an overall yield of about 80% to about 99%, about 81% to about 99%, about 82% to about 99%, about 83% to about 99%, about 84% to about 99%, about 85% to about 99%, about 86% to about 99%, about 87% to about 99%, about 88% to about 99%, about 89% to about 99%, or about 90% to about 99%.

In some embodiments, the compound of Formula I-A

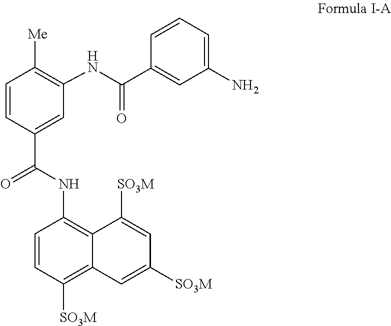

is prepared from the compound of Formula I-B

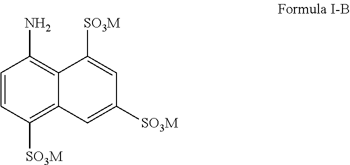

in four synthetic steps.

In some embodiments, the first synthetic step comprises contacting the compound of Formula I-B

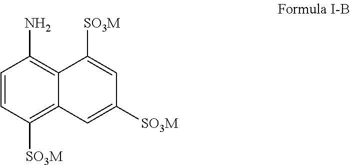

with a compound of Formula I-C

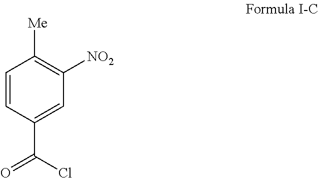 [SMILES: Cc1ccc(C(=O)Cl)cc1[N+](=O)[O-]]

in the presence of a base and a solvent to provide a compound of Formula I-D

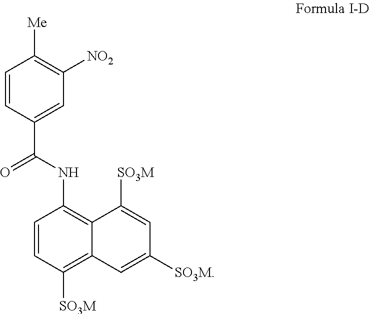

In some embodiments, the base is selected from sodium hydroxide, potassium carbonate, sodium carbonate, sodium bicarbonate, piperidine, 1,8-diazabicyclo[5.4.0]undec-7-ene, NN-diisopropylethylamine, and triethylamine. In some embodiments, the base is sodium carbonate.

In some embodiments, the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether, or a mixture thereof. In some embodiments, the solvent comprises a mixture of a first solvent and a second solvent. In some embodiments, the first solvent is a nonpolar solvent and the second solvent is a polar protic solvent. In some embodiments, the first solvent is toluene and the second solvent is water.

In some embodiments, the second synthetic step comprises contacting the compound of Formula I-D

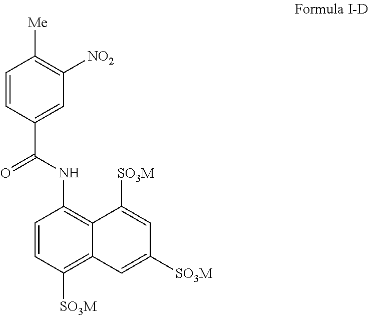

with gaseous hydrogen in the presence of a catalyst and a solvent to provide a compound of Formula I-E

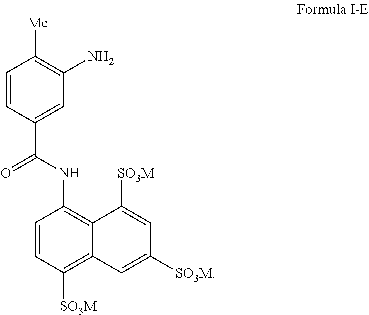

In some embodiments, the catalyst is selected from Pd/C, Pd(OH)₂, Pd/Al₂O₃, Pd(OAc)₂/Et₃SiH, (PPh₃)₃RhCl, and PtO₂. In some embodiments, the catalyst is Pd/C.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water.

In some embodiments, the third synthetic step comprises contacting the compound of Formula I-E

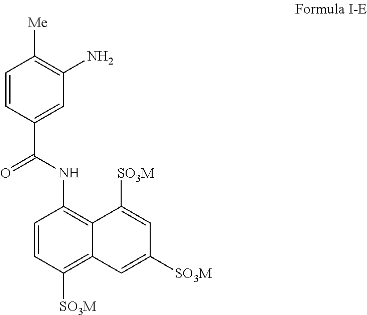

with a compound of Formula I-F

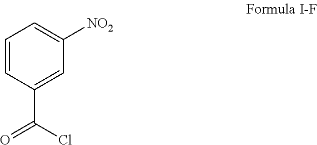 [SMILES: O=C(Cl)c1cccc([N+](=O)[O-])c1]

in the presence of a base and a solvent to provide a compound of Formula I-G

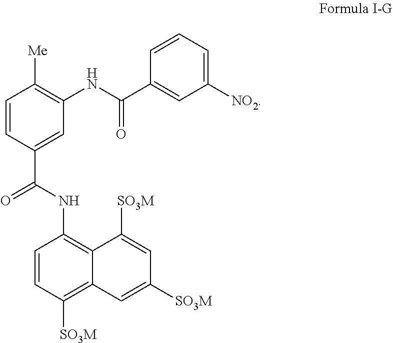

In some embodiments, the base is selected from sodium hydroxide, potassium carbonate, sodium carbonate, sodium bicarbonate, piperidine, 1,8-diazabicyclo[5.4.0]undec-7-ene, NN-diisopropylethylamine, and triethylamine. In some embodiments, the base is sodium carbonate.

In some embodiments, the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether, or a mixture thereof. In some embodiments, the solvent comprises a mixture of a first solvent and a second solvent. In some embodiments, the first solvent is a nonpolar solvent and the second solvent is a polar protic solvent. In some embodiments, the first solvent is toluene and the second solvent is water.

In some embodiments, the fourth synthetic step comprises contacting the compound of Formula I-G

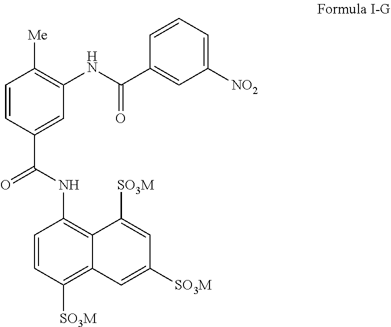

with gaseous hydrogen in the presence of a catalyst and a solvent to provide a compound of Formula I-A

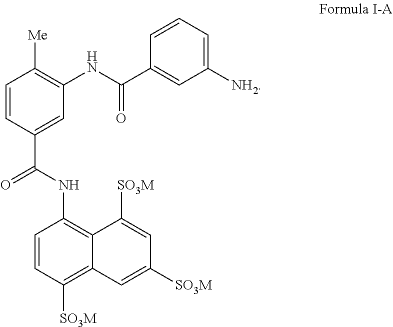

In some embodiments, the catalyst is selected from Pd/C, Pd(OH)₂, Pd/Al₂O₃, Pd(OAc)₂/Et₃SiH, (PPh₃)₃RhCl, and PtO₂. In some embodiments, the catalyst is Pd/C.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water.

In some embodiments, the crude product of each synthetic step is carried forward to the next synthetic step without purification.

In some embodiments, the final product is purified by trituration. In some embodiments, the trituration is performed with a mixture of a first solvent and a second solvent. In some embodiments, the first solvent is a polar protic solvent and the second solvent is a polar protic solvent. In some embodiments, the first solvent is ethanol and the second solvent is methanol. In some embodiments, the mixture of solvents is 30% ethanol in methanol.

In some embodiments of a compound any one of Formulae (I-A), (I-B), (I-D), (I-E), and (I-G), M is each independently H, Li, Na, or K. In some embodiments of a compound any one of Formulae (I-A), (I-B), (I-D), (I-E), and (I-G), M is each independently H, Na, or K. In some embodiments of a compound any one of Formulae (I-A), (I-B), (I-D), (I-E), and (I-G), M is each independently H, Li, or K. In some embodiments of a compound any one of Formulae (I-A), (I-B), (I-D), (I-E), and (I-G), M is each independently H, Na, or Li. In some embodiments of a compound any one of Formulae (I-A), (I-B), (I-D), (I-E), and (I-G), M is each independently H or Na. In some embodiments of a compound any one of Formulae (I-A), (I-B), (I-D), (I-E), and (I-G), M is each independently H or K. In some embodiments of a compound any one of Formulae (I-A), (I-B), (I-D), (I-E), and (I-G), M is each independently H or Li. In some embodiments of a compound any one of Formulae (I-A), (I-B), (I-D), (I-E), and (I-G), M is Na. In some embodiments of a compound any one of Formulae (I-A), (I-B), (I-D), (I-E), and (I-G), M is H. In some embodiments of a compound any one of Formulae (I-A), (I-B), (I-D), (I-E), and (I-G), M is Li. In some embodiments of a compound any one of Formulae (I-A), (I-B), (I-D), (I-E), and (I-G), M is K.

### DETAILED DESCRIPTION

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “compound” is meant to include all stereoisomers (e.g., enantiomers and diastereomers), geometric iosomers, tautomers, and isotopes of the structures depicted. Compounds herein identified by name or structure as one particular tautomeric form are intended to include other tautomeric forms unless otherwise specified.

As used herein, the term “synthetic yield” refers to the molar yield of the synthetic product relative to the limiting reagent.

As used herein, the term “synthetic step” refers to a single chemical reaction that transforms a starting material to a product. The product of the reaction does not need to be isolated or purified in order for the reaction to constitute a synthetic step.

As used herein, “SO₃Na” represents an ionic bond between an SO₃⁻ anion and a Na⁺ cation. Similarly, “SO₃Li” represents an ionic bond between an SO₃⁻ anion and a Li⁺ cation, and “SO₃K” represents an ionic bond between an SO₃⁻ anion and a K⁺ cation.

### Synthetic Methods

In one aspect, the present disclosure provides a pharmaceutical composition comprising a substantially pure composition of a compound of Formula I:

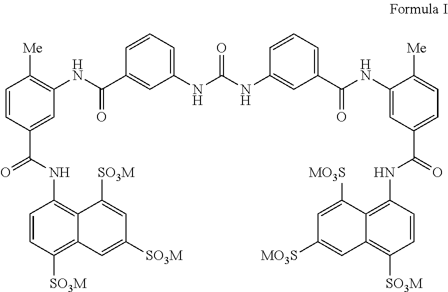

and a pharmaceutically acceptable excipient, wherein M is each independently H, Li, Na, or K. In some embodiments, M is each independently H, Na, or K. In some embodiments, M is each independently H, Li, or K. In some embodiments, M is each independently H, Na, or Li. In some embodiments, M is each independently H or Na. In some embodiments, M is each independently H or K. In some embodiments, M is each independently H or Li. In some embodiments, M is Na. In some embodiments, M is H. In some embodiments, M is Li. In some embodiments, M is K.

In some embodiments, the substantially pure composition of the compound of Formula I comprises, by weight or by mole, at least about 90%, at least about 91%, at least about 92%, at least about 93%, at least about 94%, at least about 95%, at least about 96%, at least about 97%, at least about 98%, at least about 99%, at least about 99.5%, or at least about 99.7% of the compound of Formula I. In some embodiments, the substantially pure composition of the compound of Formula I comprises, by weight or by mole, at least about 90% of the compound of Formula I. In some embodiments, the substantially pure composition of the compound of Formula I comprises, by weight or by mole, at least about 95% of the compound of Formula I. In some embodiments, the substantially pure composition of the compound of Formula I comprises, by weight or by mole, at least about 96% of the compound of Formula I. In some embodiments, the substantially pure composition of the compound of Formula I comprises, by weight or by mole, at least about 97% of the compound of Formula I. In some embodiments, the substantially pure composition of the compound of Formula I comprises, by weight or by mole, at least about 98% of the compound of Formula I. In some embodiments, the substantially pure composition of the compound of Formula I comprises, by weight or by mole, at least about 99% of the compound of Formula I. In some embodiments, the substantially pure composition of the compound of Formula I comprises, by weight or by mole, at least about 99.5% of the compound of Formula I. In some embodiments, the substantially pure composition of the compound of Formula I comprises, by weight or by mole, at least about 99.7% of the compound of Formula I.

In some embodiments, the substantially pure composition of the compound of Formula I comprises, by weight or by mole, about 95% to about 99.9%, about 96% to about 99.9%, about 97% to about 99.9%, about 98% to about 99.9%, or about 99% to about 99.9% of the compound of Formula I. In some embodiments, the substantially pure composition of the compound of Formula I comprises, by weight or by mole, about 95% to about 99.99%, about 96% to about 99.99%, about 97% to about 99.99%, about 98% to about 99.99%, or about 99% to about 99.99% of the compound of Formula I.

In some embodiments, the substantially pure composition of the compound of Formula I comprises an impurity of Formula I-A

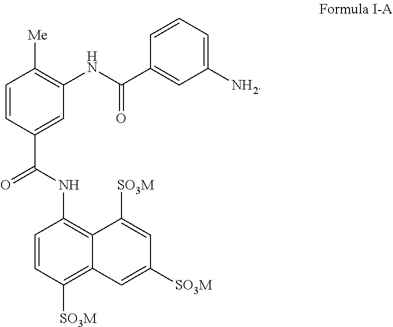

In some embodiments of an impurity of Formula I-A, M is each independently H, Li, Na, or K. In some embodiments of an impurity of Formula I-A, M is each independently H, Na, or K. In some embodiments of an impurity of Formula I-A, M is each independently H, Li, or K. In some embodiments of an impurity of Formula I-A, M is each independently H, Na, or Li. In some embodiments of an impurity of Formula I-A, M is each independently H or Na. In some embodiments of an impurity of Formula I-A, M is each independently H or K. In some embodiments of an impurity of Formula I-A, M is each independently H or Li. In some embodiments of an impurity of Formula I-A, M is Na. In some embodiments of an impurity of Formula I-A, M is H. In some embodiments, M is Li. In some embodiments of an impurity of Formula I-A, M is K.

In some embodiments, the substantially pure composition of the compound of Formula I comprises, by weight or by mole, less than about 10%, less than about 9%, less than about 8%, less than about 7%, less than about 6%, less than about 5%, less than about 4%, less than about 3%, less than about 2%, less than about 1%, less than about 0.9%, less than about 0.8%, less than about 0.7%, less than about 0.6%, or less than about 0.5% of the impurity of Formula I-A. In some embodiments, the substantially pure composition of the compound of Formula I comprises, by weight or by mole, less than about 10% of the impurity of Formula I-A. In some embodiments, the substantially pure composition of the compound of Formula I comprises, by weight or by mole, less than about 5% of the impurity of Formula I-A. In some embodiments, the substantially pure composition of the compound of Formula I comprises, by weight or by mole, less than about 4% of the impurity of Formula I-A. In some embodiments, the substantially pure composition of the compound of Formula I comprises, by weight or by mole, less than about 3% of the impurity of Formula I-A. In some embodiments, the substantially pure composition of the compound of Formula I comprises, by weight or by mole, less than about 2% of the impurity of Formula I-A. In some embodiments, the substantially pure composition of the compound of Formula I comprises, by weight or by mole, less than about 1% of the impurity of Formula I-A. In some embodiments, the substantially pure composition of the compound of Formula I comprises, by weight or by mole, less than about 0.5% of the impurity of Formula I-A.

In some embodiments, the substantially pure composition of the compound of Formula I comprises, by weight or by mole, about 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, or about 0.01% to about 0.5% of the impurity of Formula I-A.

In some embodiments, the substantially pure composition of the compound of Formula I comprises, by weight or by mole, about 0.005% to about 10%, 0.005% to about 9%, 0.005% to about 8%, 0.005% to about 7%, 0.005% to about 6%, 0.005% to about 5%, 0.005% to about 4%, 0.005% to about 3%, 0.005% to about 2%, 0.005% to about 1%, or 0.005% to about 0.5% of the impurity of Formula I-A.

In some embodiments, the substantially pure composition of the compound of Formula I comprises, by weight or by mole, about 0.001% to about 10%, about 0.001% to about 9%, about to about 8%, about 0.001% to about 7%, about 0.001% to about 6%, about 0.001% to about 5%, about 0.001% to about 4%, about 0.001% to about 3%, about 0.001% to about 2%, about to about 1%, or about 0.001% to about 0.5% of the impurity of Formula I-A.

In some embodiments, the pharmaceutically acceptable excipient is selected from an adjuvant, carrier, glidant, sweetening agent, diluent, preservative, dye, colorant, flavor enhancer, surfactant, wetting agent, dispersing agent, suspending agent, stabilizer, isotonic agent, solvent, or emulsifier. In some embodiments, the pharmaceutically acceptable excipient is an adjuvant. In some embodiments, the pharmaceutically acceptable excipient is a carrier. In some embodiments, the pharmaceutically acceptable excipient is a glidant. In some embodiments, the pharmaceutically acceptable excipient is a sweetening agent. In some embodiments, the pharmaceutically acceptable excipient is a diluent. In some embodiments, the pharmaceutically acceptable excipient is a preservative. In some embodiments, the pharmaceutically acceptable excipient is a dye. In some embodiments, the pharmaceutically acceptable excipient is a colorant. In some embodiments, the pharmaceutically acceptable excipient is a flavor enhancer. In some embodiments, the pharmaceutically acceptable excipient is a surfactant. In some embodiments, the pharmaceutically acceptable excipient is a wetting agent. In some embodiments, the pharmaceutically acceptable excipient is a dispersing agent. In some embodiments, the pharmaceutically acceptable excipient is a suspending agent. In some embodiments, the pharmaceutically acceptable excipient is a stabilizer. In some embodiments, the pharmaceutically acceptable excipient is an isotonic agent. In some embodiments, the pharmaceutically acceptable excipient is a solvent. In some embodiments, the pharmaceutically acceptable excipient is an emulsifier.

In another aspect, the present disclosure provides a method of treating an autism spectrum disorder in a subject in need thereof, wherein the method comprises administering to the subject a therapeutically effective amount of the pharmaceutical composition disclosed herein. In some embodiments, the pharmaceutical composition is administered to the subject intravenously, intranasally, subcutaneously, or parenterally. In some embodiments, the pharmaceutical composition is administered to the subject intravenously. In some embodiments, the pharmaceutical composition is administered to the subject subcutaneously. In some embodiments, the pharmaceutical composition is administered to the subject parenterally.

In another aspect, the present disclosure provides a method of preparing a compound of Formula I-A

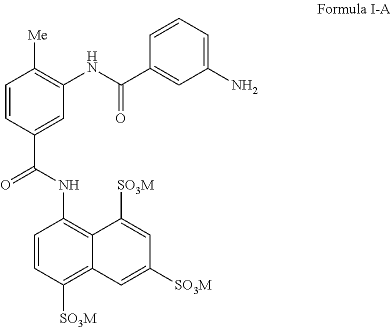

from a compound of Formula I-B

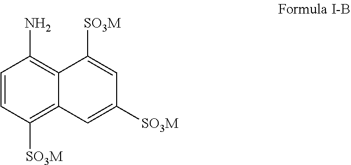

wherein M is each independently H, Li, Na, or K, and wherein the method provides the compound of Formula I-A in an overall yield of greater than about 80%. In some embodiments of a compound of Formula I-B, M is each independently H, Na, or K. In some embodiments of a compound of Formula I-B, M is each independently H, Li, or K. In some embodiments of a compound of Formula I-B, M is each independently H, Na, or Li. In some embodiments of a compound of Formula I-B, M is each independently H or Na. In some embodiments of a compound of Formula I-B, M is each independently H or K. In some embodiments of a compound of Formula I-B, M is each independently H or Li. In some embodiments of a compound of Formula I-B, M is Na. In some embodiments of a compound of Formula I-B, M is H. In some embodiments of a compound of Formula I-B, M is Li. In some embodiments of a compound of Formula I-B, M is K.

In some embodiments, the method provides the compound of Formula I-A in an overall yield of greater than about 81%, greater than about 82%, greater than about 83%, greater than about 84%, greater than about 85%, greater than about 86%, greater than about 87%, greater than about 88%, greater than about 89%, greater than about 90%, greater than about 91%, greater than about 92%, greater than about 93%, greater than about 94%, greater than about 95%, greater than about 96%, greater than about 97%, greater than about 98%, greater than about 99%, or greater than about 99.5%. In some embodiments, the method provides the compound of Formula I-A in an overall yield of greater than about 90%. In some embodiments, the method provides the compound of Formula I-A in an overall yield of greater than about 95%. In some embodiments, the method provides the compound of Formula I-A in an overall yield of greater than about 96%. In some embodiments, the method provides the compound of Formula I-A in an overall yield of greater than about 97%. In some embodiments, the method provides the compound of Formula I-A in an overall yield of greater than about 98%. In some embodiments, the method provides the compound of Formula I-A in an overall yield of greater than about 99%. In some embodiments, the method provides the compound of Formula I-A in an overall yield of greater than about 99.5%. In some embodiments, the method provides the compound of Formula I-A in an overall yield of about 80% to about 99%, about 81% to about 99%, about 82% to about 99%, about 83% to about 99%, about 84% to about 99%, about 85% to about 99%, about 86% to about 99%, about 87% to about 99%, about 88% to about 99%, about 89% to about 99%, or about 90% to about 99%. In some embodiments, the method provides the compound of Formula I-A in an overall yield of about 80% to about 99.9%, about 81% to about 99.9%, about 82% to about 99.9%, about 83% to about 99.9%, about 84% to about 99.9%, about 85% to about 99.9%, about 86% to about 99.9%, about 87% to about 99.9%, about 88% to about 99.9%, about 89% to about 99.9%, or about 90% to about 99.9%. In some embodiments, the method provides the compound of Formula I-A in an overall yield of about 80% to about 99.99%, about 81% to about 99.99%, about 82% to about 99.99%, about 83% to about 99.99%, about 84% to about 99.99%, about 85% to about 99.99%, about 86% to about 99.99%, about 87% to about 99.99%, about 88% to about 99.99%, about 89% to about 99.99%, or about 90% to about 99.99%.

In some embodiments, the compound of Formula I-A

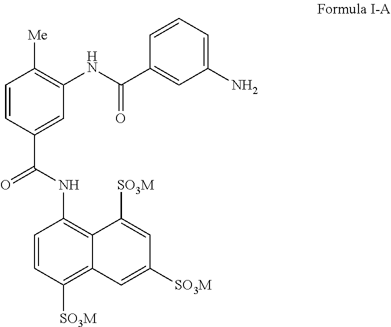

is prepared from the compound of Formula I-B

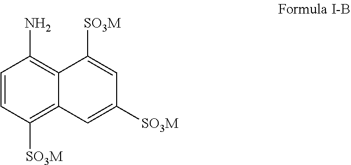

in four synthetic steps.

In some embodiments, the first synthetic step comprises contacting the compound of Formula I-B

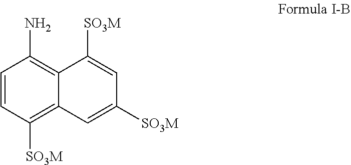

with a compound of Formula I-C

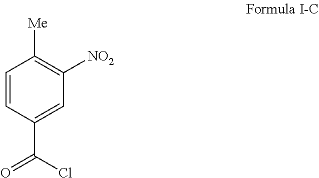 [SMILES: Cc1ccc(C(=O)Cl)cc1[N+](=O)[O-]]

in the presence of a base and a solvent to provide a compound of Formula I-D

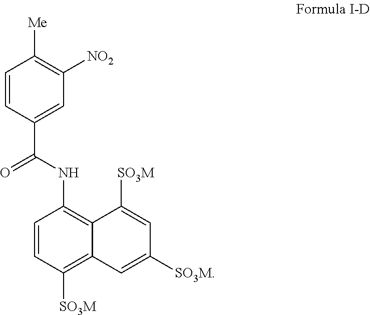

In some embodiments, the base is selected from sodium hydroxide, potassium carbonate, sodium carbonate, sodium bicarbonate, piperidine, 1,8-diazabicyclo[5.4.0]undec-7-ene, NN-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine.

In some embodiments, the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether, or a mixture thereof. In some embodiments, the solvent comprises water. In some embodiments, the solvent comprises ethyl acetate. In some embodiments, the solvent comprises dichloromethane. In some embodiments, the solvent comprises tetrahydrofuran. In some embodiments, the solvent comprises diethyl ether. In some embodiments, the solvent comprises dimethylformamide. In some embodiments, the solvent comprises dimethylsulfoxide. In some embodiments, the solvent comprises methanol. In some embodiments, the solvent comprises ethanol. In some embodiments, the solvent comprises acetone. In some embodiments, the solvent comprises acetonitrile. In some embodiments, the solvent comprises 1,4-dioxane. In some embodiments, the solvent comprises hexane. In some embodiments, the solvent comprises methyl tert-butyl ether. In some embodiments, the solvent comprises a mixture of a first solvent and a second solvent. In some embodiments, the first solvent is a nonpolar solvent. In some embodiments, the first solvent is a polar aprotic solvent. In some embodiments, the first solvent is a polar protic solvent. In some embodiments, the second solvent is a nonpolar solvent. In some embodiments, the second solvent is a polar aprotic solvent. In some embodiments, the second solvent is a polar protic solvent. In some embodiments, the first solvent is a nonpolar solvent and the second solvent is a polar protic solvent. In some embodiments, the first solvent is toluene and the second solvent is water.

In some embodiments, the second synthetic step comprises subjecting the compound of Formula I-D

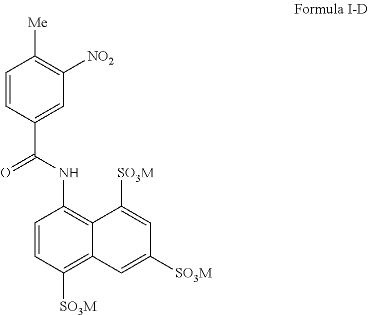

to a reducing step to provide a compound of Formula I-E

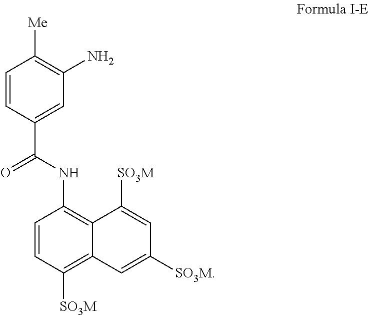

In some embodiments, the reducing step comprises subjecting the compound of Formula I-D to a catalytic hydrogenation. In some embodiments, the reducing step comprises treating the compound of Formula I-D with iron and an acid. In some embodiments, the reducing step comprises treating the compound of Formula I-D with sodium hydrosulfite. In some embodiments, the reducing step comprises treating the compound of Formula I-D with sodium sulfide. In some embodiments, the reducing step comprises treating the compound of Formula I-D with tin(II) chloride. In some embodiments, the reducing step comprises treating the compound of Formula I-D with titanium(III) chloride. In some embodiments, the reducing step comprises treating the compound of Formula I-D with samarium. In some embodiments, the reducing step comprises treating the compound of Formula I-D with hydroiodic acid.

In some embodiments, the second synthetic step comprises contacting the compound of Formula I-D

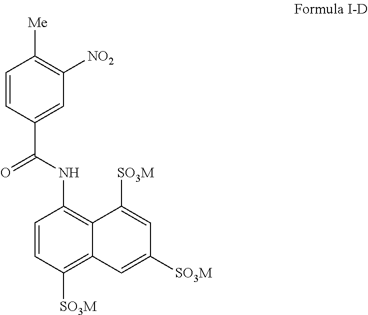

with gaseous hydrogen in the presence of a catalyst and a solvent to provide a compound of Formula I-E

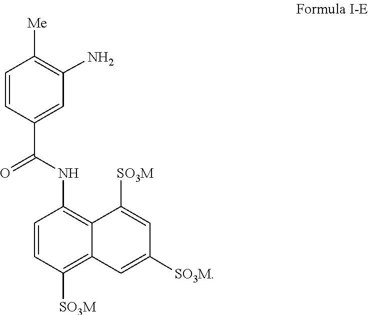

In some embodiments, the catalyst is selected from Pd/C, Pd(OH)₂, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the third synthetic step comprises contacting the compound of Formula I-E

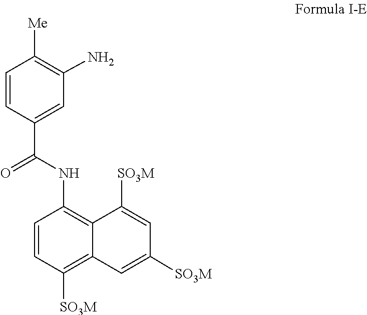

with a compound of Formula I-F

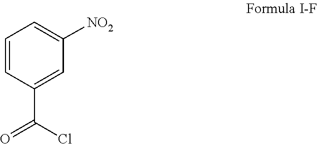 [SMILES: O=C(Cl)c1cccc([N+](=O)[O-])c1]

in the presence of a base and a solvent to provide a compound of Formula I-G

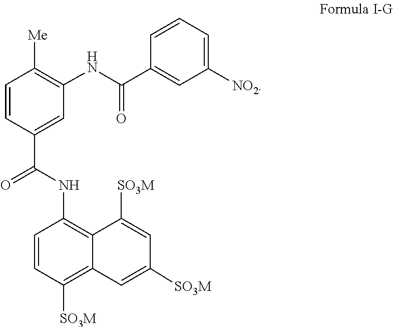

In some embodiments, the base is selected from sodium hydroxide, potassium carbonate, sodium carbonate, sodium bicarbonate, piperidine, 1,8-diazabicyclo[5.4.0]undec-7-ene, NN-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine.

In some embodiments, the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether or a mixture thereof. In some embodiments, the solvent comprises water. In some embodiments, the solvent comprises ethyl acetate. In some embodiments, the solvent comprises dichloromethane. In some embodiments, the solvent comprises tetrahydrofuran. In some embodiments, the solvent comprises diethyl ether. In some embodiments, the solvent comprises dimethylformamide. In some embodiments, the solvent comprises dimethylsulfoxide. In some embodiments, the solvent comprises methanol. In some embodiments, the solvent comprises ethanol. In some embodiments, the solvent comprises acetone. In some embodiments, the solvent comprises acetonitrile. In some embodiments, the solvent comprises 1,4-dioxane. In some embodiments, the solvent comprises hexane. In some embodiments, the solvent comprises methyl tert-butyl ether. In some embodiments, the solvent comprises a mixture of a first solvent and a second solvent. In some embodiments, the first solvent is a nonpolar solvent. In some embodiments, the first solvent is a polar aprotic solvent. In some embodiments, the first solvent is a polar protic solvent. In some embodiments, the second solvent is a nonpolar solvent. In some embodiments, the second solvent is a polar aprotic solvent. In some embodiments, the second solvent is a polar protic solvent. In some embodiments, the first solvent is a nonpolar solvent and the second solvent is a polar protic solvent. In some embodiments, the first solvent is toluene and the second solvent is water.

In some embodiments, the fourth synthetic step comprises subjecting the compound of Formula I-G

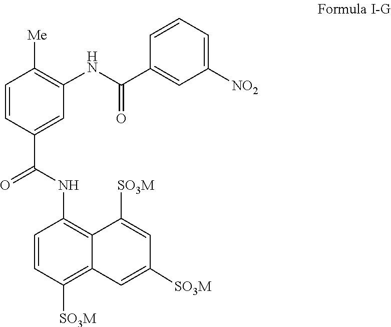

to a reducing step to provide a compound of Formula I-A

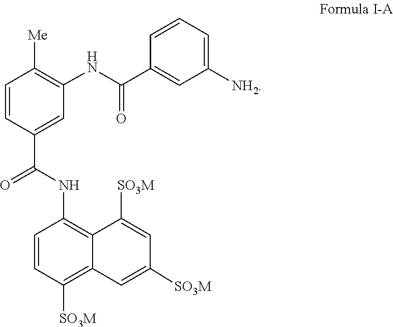

In some embodiments, the reducing step comprises subjecting the compound of Formula I-D to a catalytic hydrogenation. In some embodiments, the reducing step comprises treating the compound of Formula I-D with iron and an acid. In some embodiments, the reducing step comprises treating the compound of Formula I-D with sodium hydrosulfite. In some embodiments, the reducing step comprises treating the compound of Formula I-D with sodium sulfide. In some embodiments, the reducing step comprises treating the compound of Formula I-D with tin(II) chloride. In some embodiments, the reducing step comprises treating the compound of Formula I-D with titanium(III) chloride. In some embodiments, the reducing step comprises treating the compound of Formula I-D with samarium. In some embodiments, the reducing step comprises treating the compound of Formula I-D with hydroiodic acid.

In some embodiments, the fourth synthetic step comprises contacting the compound of Formula I-G

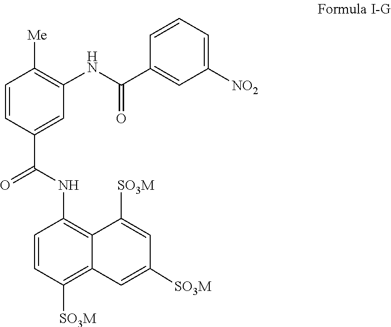

with gaseous hydrogen in the presence of a catalyst and a solvent to provide a compound of Formula I-A

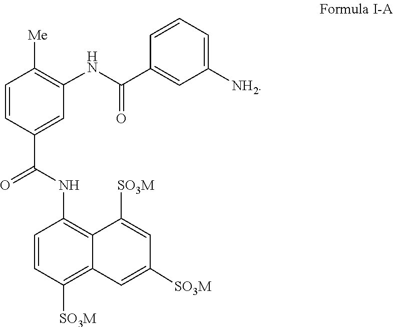

In some embodiments, the catalyst is selected from Pd/C, Pd(OH)₂, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the crude product of each synthetic step is carried forward to the next synthetic step without purification.

In some embodiments, the final product is purified by recrystallization. In some embodiments, the final product is purified by trituration. In some embodiments, the trituration is performed with a single solvent. In some embodiments, the solvent is methanol. In some embodiments, the solvent is ethanol. In some embodiments, the trituration is performed with a mixture of a first solvent and a second solvent. In some embodiments, the first solvent is a nonpolar solvent. In some embodiments, the first solvent is a polar aprotic solvent. In some embodiments, the first solvent is a polar protic solvent. In some embodiments, the second solvent is a nonpolar solvent. In some embodiments, the second solvent is a polar aprotic solvent. In some embodiments, the second solvent is a polar protic solvent. In some embodiments, the first solvent is a polar protic solvent and the second solvent is a polar protic solvent. In some embodiments, the first solvent is ethanol and the second solvent is methanol. In some embodiments, the mixture of solvents is 10% ethanol in methanol. In some embodiments, the mixture of solvents is 20% ethanol in methanol. In some embodiments, the mixture of solvents is 30% ethanol in methanol. In some embodiments, the mixture of solvents is 40% ethanol in methanol. In some embodiments, the mixture of solvents is 50% ethanol in methanol. In some embodiments, the mixture of solvents is 60% ethanol in methanol. In some embodiments, the mixture of solvents is 70% ethanol in methanol. In some embodiments, the mixture of solvents is 80% ethanol in methanol. In some embodiments, the mixture of solvents is 90% ethanol in methanol.

In some embodiments of a compound any one of Formulae (I), (I-A), (I-B), (I-D), (I-E), and (I-G), M is each independently H, Li, Na, or K. In some embodiments of a compound any one of Formulae (I), (I-A), (I-B), (I-D), (I-E), and (I-G), M is each independently H, Na, or K. In some embodiments of a compound any one of Formulae (I), (I-A), (I-B), (I-D), (I-E), and (I-G), M is each independently H, Li, or K. In some embodiments of a compound any one of Formulae (I), (I-A), (I-B), (I-D), (I-E), and (I-G), M is each independently H, Na, or Li. In some embodiments of a compound any one of Formulae (I), (I-A), (I-B), (I-D), (I-E), and (I-G), M is each independently H or Na. In some embodiments of a compound any one of Formulae (I), (I-A), (I-B), (I-D), (I-E), and (I-G), M is each independently H or K. In some embodiments of a compound any one of Formulae (I), (I-A), (I-B), (I-D), (I-E), and (I-G), M is each independently H or Li. In some embodiments of a compound any one of Formulae (I), (I-A), (I-B), (I-D), (I-E), and (I-G), M is Na. In some embodiments of a compound any one of Formulae (I), (I-A), (I-B), (I-D), (I-E), and (I-G), M is H. In some embodiments of a compound any one of Formulae (I), (I-A), (I-B), (I-D), (I-E), and (I-G), M is Li. In some embodiments of a compound any one of Formulae (I), (I-A), (I-B), (I-D), (I-E), and (I-G), M is K.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.
Embodiment 1. A pharmaceutical composition comprising a substantially pure composition of a compound of Formula I:

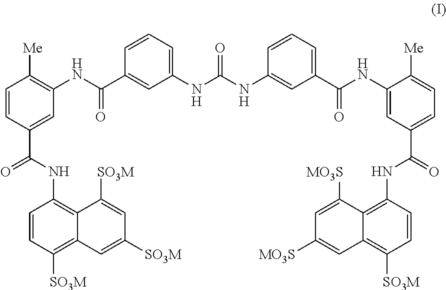

and a pharmaceutically acceptable excipient, wherein M is each independently H, Li, Na, or K (optionally wherein M is Na).
Embodiment 2. The pharmaceutical composition of embodiment 1, wherein the substantially pure composition of the compound of Formula I comprises, by weight or by mole, at least about 95%, at least about 96%, or at least about 97% of the compound of Formula I.
Embodiment 3. The pharmaceutical composition of embodiment 2, wherein the substantially pure composition of the compound of Formula I comprises, by weight or by mole, about 95% to about 99.9%, about 96% to about 99.9%, or about 97% to about 99.9% of the compound of Formula I.
Embodiment 4. The pharmaceutical composition of any one of embodiments 1 to 3, wherein the substantially pure composition of the compound of Formula I comprises an impurity of Formula I-A

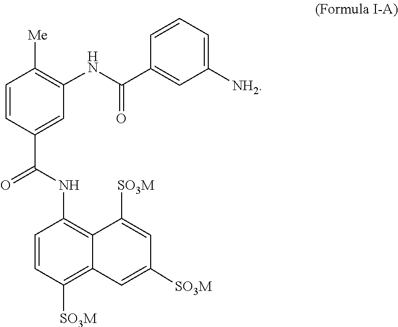

Embodiment 5. The pharmaceutical composition of embodiment 4, wherein the substantially pure composition of the compound of Formula I comprises, by weight or by mole, less than about 5%, less than about 4%, or less than about 3% of the impurity of Formula I-A.
Embodiment 6. The pharmaceutical composition of embodiment 5, wherein the substantially pure composition of the compound of Formula I comprises, by weight or by mole, about 0.01% to about 5%, about 0.01% to about 4%, or about 0.01% to about 3% of the impurity of Formula I-A.
Embodiment 7. A method of treating an autism spectrum disorder (ASD) in a subject in need thereof, wherein the method comprises administering to the subject a therapeutically effective amount of the pharmaceutical composition of any one of embodiments 1 to 6.
Embodiment 8. The method of embodiment 7, wherein the pharmaceutical composition is administered to the subject intravenously, intranasally, subcutaneously, or parenterally.
Embodiment 9. The method of embodiment 8, wherein the pharmaceutical composition is administered to the subject intravenously.
Embodiment 10. A method of preparing a compound of Formula I-A

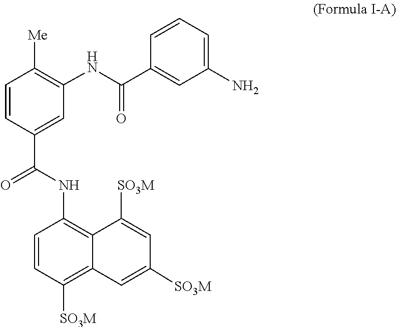

from a compound of Formula I-B

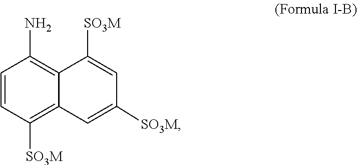

wherein M is each independently H, Li, Na, or K (optionally wherein M is Na), and wherein the method provides the compound of Formula I-A in an overall yield of greater than 80%, 81%, 82%, 83%, 84%, 85%, 86%, 87%, 88%, 89%, or 90%.
Embodiment 11. The method of embodiment 10, wherein the method provides the compound of Formula I-A in an overall yield of greater than 90%.
Embodiment 12. The method of embodiment 10 or 11, wherein the compound of Formula I-A

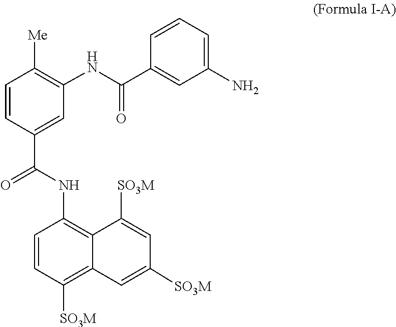

is prepared from the compound of Formula I-B

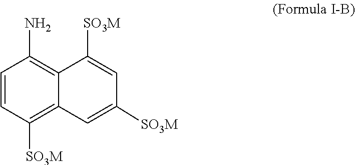

in four synthetic steps.
Embodiment 13. The method of embodiment 12, wherein the first synthetic step comprises contacting the compound of Formula I-B

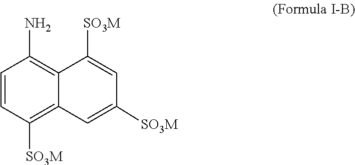

with a compound of Formula I-C

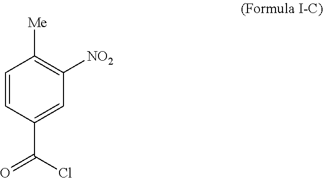 [SMILES: Cc1ccc(C(=O)Cl)cc1[N+](=O)[O-]]

in the presence of a base and a solvent to provide a compound of Formula I-D

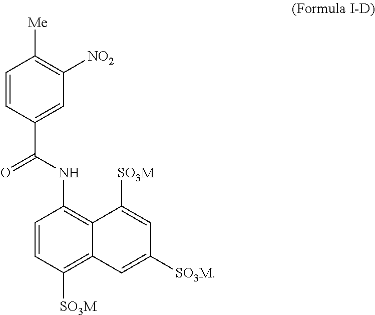

Embodiment 14. The method of embodiment 13, wherein the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine.
Embodiment 15. The method of embodiment 14, wherein the base is sodium carbonate.
Embodiment 16. The method of embodiment 13, wherein the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether, or a mixture thereof.
Embodiment 17. The method of embodiment 16, wherein the solvent comprises a mixture of a first solvent and a second solvent.
Embodiment 18. The method of embodiment 17, wherein the first solvent is a nonpolar solvent and the second solvent is a polar protic solvent.
Embodiment 19. The method of embodiment 18, wherein the first solvent is toluene and the second solvent is water.
Embodiment 20. The method of any one of embodiments 13-19, wherein the second synthetic step comprises contacting the compound of Formula I-D

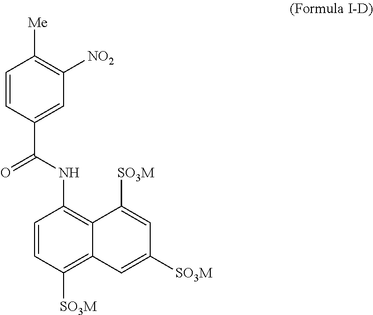

with gaseous hydrogen in the presence of a catalyst and a solvent to provide a compound of Formula I-E

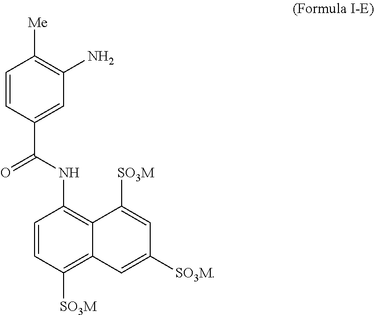

Embodiment 21. The method of embodiment 20, wherein the catalyst is selected from Pd/C, Pd(OH)₂, Pd/Al₂O₃, Pd(OAc)₂/Et₃SiH, (PPh₃)₃RhCl, and PtO₂.
Embodiment 22. The method of embodiment 21, wherein the catalyst is Pd/C.
Embodiment 23. The method of embodiment 20, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.
Embodiment 24. The method of embodiment 23, wherein the solvent is water.
Embodiment 25. The method of any one of embodiments 20-24, wherein the third synthetic step comprises contacting the compound of Formula I-E

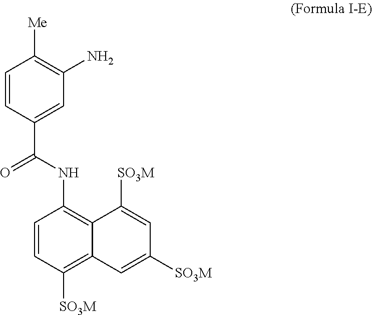

with a compound of Formula I-F

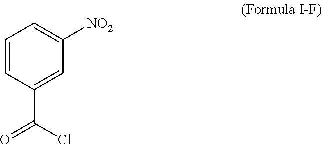 [SMILES: O=C(Cl)c1cccc([N+](=O)[O-])c1]

in the presence of a base and a solvent to provide a compound of Formula I-G

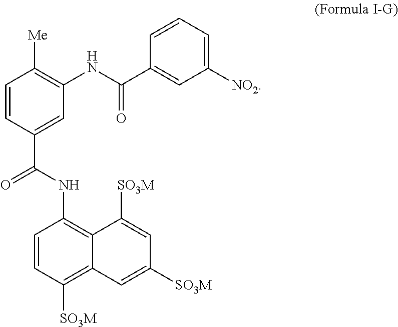

Embodiment 26. The method of embodiment 25, wherein the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine.
Embodiment 27. The method of embodiment 26, wherein the base is sodium carbonate.
Embodiment 28. The method of embodiment 25, wherein the solvent comprises water, ethyl acetate, dichloromethane, tetrahydrofuran, diethyl ether, dimethylformamide, dimethylsulfoxide, methanol, ethanol, acetone, acetonitrile, 1,4-dioxane, hexane, methyl tert-butyl ether, or a mixture thereof.
Embodiment 29. The method of embodiment 28, wherein the solvent comprises a mixture of a first solvent and a second solvent.
Embodiment 30. The method of embodiment 29, wherein the first solvent is a nonpolar solvent and the second solvent is a polar protic solvent.
Embodiment 31. The method of embodiment 30, wherein the first solvent is toluene and the second solvent is water.
Embodiment 32. The method of any one of embodiments 25-31, wherein the fourth synthetic step comprises contacting the compound of Formula I-G

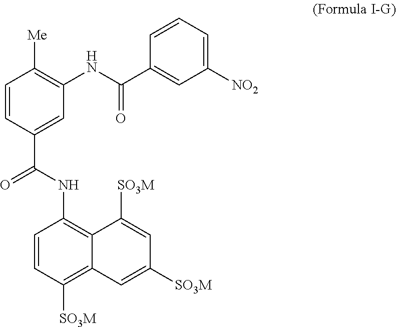

with gaseous hydrogen in the presence of a catalyst and a solvent to provide a compound of Formula I-A

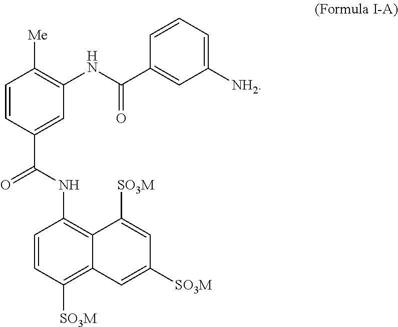

Embodiment 33. The method of embodiment 32, wherein the catalyst is selected from Pd/C, Pd(OH)₂, Pd/Al₂O₃, Pd(OAc)₂/Et₃SiH, (PPh₃)₃RhCl, and PtO₂.
Embodiment 34. The method of embodiment 33, wherein the catalyst is Pd/C.
Embodiment 35. The method of embodiment 32, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, and methyl tert-butyl ether.
Embodiment 36. The method of embodiment 35, wherein the solvent is water.
Embodiment 37. The method of any one of embodiments 12-36, wherein the crude product of each synthetic step is carried forward to the next synthetic step without purification.
Embodiment 38. The method of embodiment 37, wherein the final product is purified by trituration.
Embodiment 39. The method of embodiment 38, wherein the trituration is performed with a mixture of a first solvent and a second solvent.
Embodiment 40. The method of embodiment 39, wherein the first solvent is a polar protic solvent and the second solvent is a polar protic solvent.
Embodiment 41. The method of embodiment 40, wherein the first solvent is ethanol and the second solvent is methanol.
Embodiment 42. The method of embodiment 41, wherein the mixture of solvents is 30% ethanol in methanol.
Embodiment 43. A method of treating fragile X-associated tremor/ataxia (FXTAS) in a subject in need thereof, wherein the method comprises administering to the subject a therapeutically effective amount of the pharmaceutical composition of any one of embodiments 1 to 6.
Embodiment 44. The method of embodiment 43, wherein the pharmaceutical composition is administered to the subject intravenously, intranasally, subcutaneously, or parenterally.

### EXAMPLES

### Example 1: Preparation of Suramin

### Step 1: Preparation of sodium 8-(4-methyl-3-nitrobenzamido)naphthalene-1,3,5-trisulfonate 2

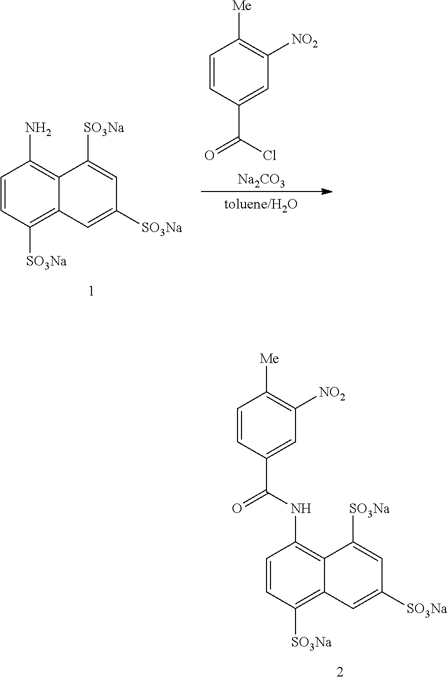

Sodium 8-aminonaphthalene-1,3,5-trisulfonate 1 (1.50 kg, 6.68 mol, 1.0 equiv) was dissolved in water (18.0 L, 0.37 M) with vigorous stirring. 4-methyl-3-nitrobenzoyl chloride (1.87 kg, 9.35 mol, 1.40 equiv) in toluene (4.50 L, 2.08 M) was added dropwise in portions. The pH of the aqueous layer was monitored by pH paper or probe and maintained above pH 2.0 via addition of 2.0 M sodium carbonate (<2.00 L, <4.00 mol). Upon complete consumption of sodium 8-aminonaphthalene-1,3,5-trisulfonate 1, the reaction mixture was transferred to a separatory funnel and the toluene layer was discarded. The aqueous layer was acidified to pH 2.0 with a 6.0 M hydrochloric acid solution and extracted three times with methyl tert-butyl ether (2.5× volumes each, 7.50 L). The pooled organic extracts were discarded. The aqueous layer was neutralized to pH 7.0 with 2.0 M sodium carbonate. The resulting aqueous solution was carried forward to the next synthetic step without further purification.

### Step 2: Preparation of sodium 8-(3-amino-4-methylbenzamido)naphthalene-1,3,5-trisulfonate 3

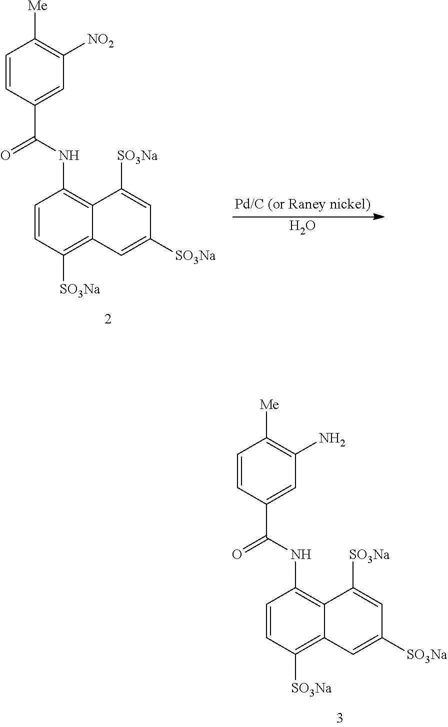

An 8.00 L Parr reactor was charged with the crude solution of sodium 8-(4-methyl-3-nitrobenzamido)naphthalene-1,3,5-trisulfonate 2 from step 1 (1.023 kg, 1.67 mol, 1.0 equiv, ˜6.5 kg of solution). The solution was treated with Pd/C (711 g, 0.334 mol, 5 mol % loading, 5 wt. % overall Pd content on wet carbon) split into four batches of 178 g Pd/C. The reactor was sealed up and connected to pressurized nitrogen and hydrogen sources. After stirring commenced, the reactor was pressurized and then vented three times with nitrogen and then three times with hydrogen. The reactor was then charged a fourth time with hydrogen to 60 psi and the reaction mixture was stirred at room temperature. The headspace pressure of the reactor was monitored to observe hydrogen uptake and the reactor was recharged with hydrogen when necessary. Upon complete consumption of the starting material, the reaction mixture was filtered through a piece of GF/F paper without allowing the surface of the filter to become dry. The resulting aqueous solution was carried forward to the next synthetic step without further purification.

### Step 3: Preparation of sodium 8-(4-methyl-3-(3-nitrobenzamido)benzamido)naphthalene-1,3,5-trisulfonate 4

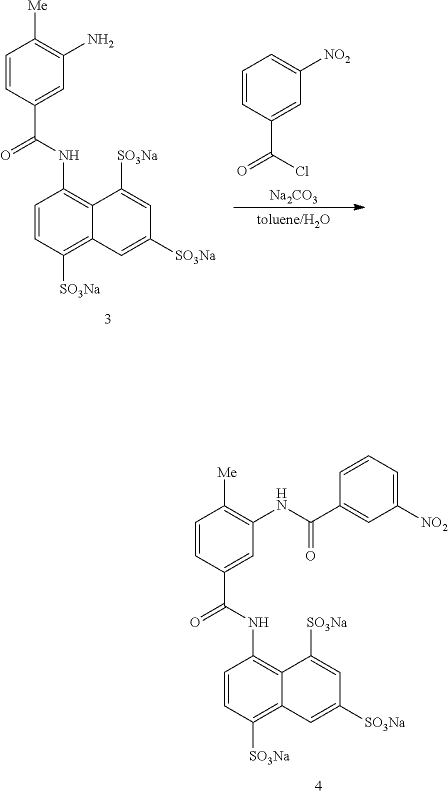

The crude solution of sodium 8-(3-amino-4-methylbenzamido)naphthalene-1,3,5-trisulfonate 3 from step 2 (3.89 kg, 6.68 mol, 1.0 equiv) in water (38.9 L, 0.17 M) treated dropwise with 3-nitrobenzoyl chloride (1.74 kg, 42.34 mol, 1.40 equiv) in toluene (4.50 L, 9.4 M). The pH of the aqueous layer was monitored by pH paper or probe and maintained above pH 2.0 via addition of 2.0 M sodium carbonate (<2.00 L, <4.00 mol). Upon complete consumption of sodium 8-(3-amino-4-methylbenzamido)naphthalene-1,3,5-trisulfonate 3, the reaction mixture was transferred to a separatory funnel and the toluene layer was discarded. The aqueous layer was acidified to pH 2.0 with a 6.0 M hydrochloric acid solution and extracted four times with methyl tert-butyl ether (2× volumes each, 6.00 L). The pooled organic extracts were discarded. The aqueous layer was neutralized to pH 7.0 with 2.0 M sodium carbonate. The resulting aqueous solution was carried forward to the next synthetic step without further purification.

### Step 4: Preparation of sodium 8-(3-(3-aminobenzamido)-4-methylbenzamido)naphthalene-1,3,5-trisulfonate 5

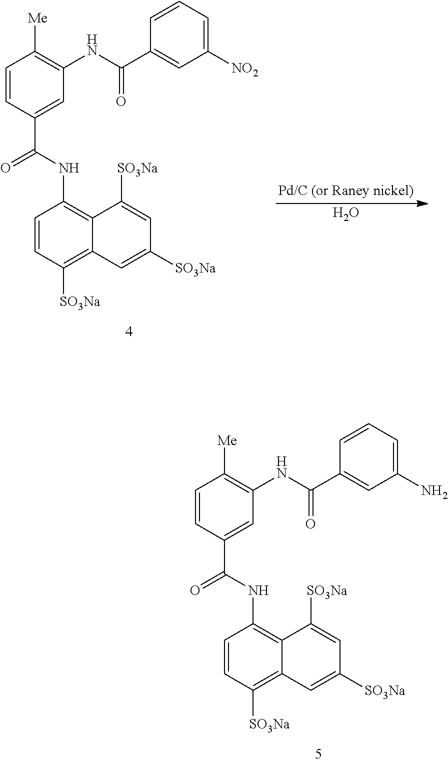

A 10.0 L Parr reactor was charged with the crude solution of sodium 8-(4-methyl-3-(3-nitrobenzamido)benzamido)naphthalene-1,3,5-trisulfonate 4 from step 1 (2.445 kg, 3.34 mol, 1.0 equiv). The solution was treated with Pd/C (355.6 g, 167 mol, 10% Pd-dry, 5% Pd-wet, 0.05 equiv). The reactor was sealed up and connected to pressurized nitrogen and hydrogen sources. After stirring commenced, the reactor was pressurized and then vented three times with nitrogen and then three times with hydrogen. The reactor was then charged a fourth time with hydrogen to 60 psi and the reaction mixture was stirred at room temperature. The headspace pressure of the reactor was monitored to observe hydrogen uptake and the reactor was recharged with hydrogen when necessary. Upon complete consumption of the starting material, the reaction mixture was filtered through a piece of GF/F paper without allowing the surface of the filter to become dry.

The resulting aqueous solution was concentrated on a rotary evaporator, redissolved in water (4.0× volumes, 18.76 L), and treated with 10 wt %-equivalent of Silicycle SiliaMetS® Thiol scavenger resin (469 g, 10 wt/wt loading, 471 g actual charge). The resulting slurry was heated to 45° C. overnight, cooled to room temperature, and filtered through a Buchner funnel lined with GF/F paper, and the filter cake was washed with water (250 mL).

The filtrate was divided into two batches of 2.35 kg for precipitation. The filtrate (11.82 kg solution, 2.35 kg sodium 8-(3-(3-aminobenzamido)-4-methylbenzamido)naphthalene-1,3,5-trisulfonate 5, 3.34 mol) was charged to a 5 liter addition funnel equipped to a 72 liter reactor charged with 38 liters of isopropyl acetate at room temperature. The aqueous solution was added to the IPA solution with vigorous stirring over 5 hours, and the resulting slurry was aged overnight. The resulting solid was isolated by vacuum filtration through a medium-fitted polypropylene table top filter funnel lined with polypropylene cloth. The filter cake was washed with 20% aqueous isopropyl acetate (3.84× volumes, 9.00 L) and then with isopropyl acetate (2.0× volumes, 4.69 L), and dried in a vacuum oven at 40° C. under a nitrogen stream for 3.5 days to afford sodium 8-(3-(3-aminobenzamido)-4-methylbenzamido)naphthalene-1,3,5-trisulfonate 5 (2.072 kg). The second batch yielded 2.222 kg of sodium 8-(3-(3-aminobenzamido)-4-methylbenzamido)naphthalene-1,3,5-trisulfonate 5 for a total yield of 4.294 kg (91.6% yield).

### Step 5: Preparation of Suramin 6

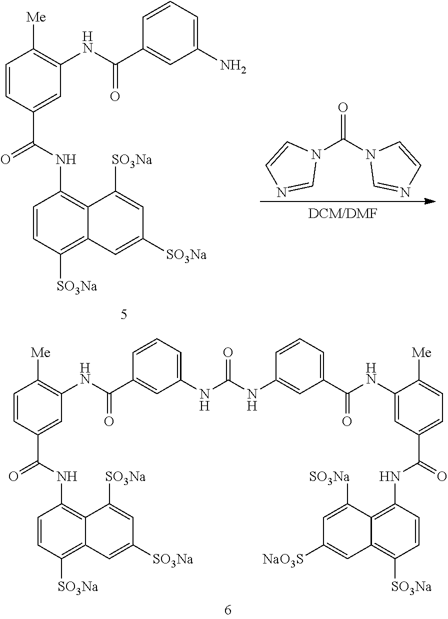

Sodium 8-(3-(3-aminobenzamido)-4-methylbenzamido)naphthalene-1,3,5-trisulfonate 5 (25.0 g, 35.63 mmol, 1.0 equiv) and imidazole hydrochloride (745 mg, 7.13 mmol, 0.20 equiv) were suspended in 4:1 acetonitrile/water (0.14 M). 1,1′-carbonyldiimidazole (6.93 g, 42.8 mmol, 1.20 equiv) was added in portions over the course of 19 hours. Upon complete consumption of sodium 8-(3-(3-aminobenzamido)-4-methylbenzamido)naphthalene-1,3,5-trisulfonate 5, the organic layer was discarded. The aqueous layer was diluted with methanol (3× volumes, 75.0 mL) and basified to pH 9.0 with sodium methoxide in methanol (1.02 mL, 4.45 mmol, 0.25 equiv). The solution was treated with Darco-60 activated carbon (5.00 g, 20 wt %-eq.) and stirred at room temperature for 30 minutes. The resulting slurry was polish-filtered (GF/F), and the filter cake was washed with methanol (1.5× volumes, 37.5 mL). The resulting solution was cooled to 5-10° C. and ethanol (12× volumes, 300 mL) was added dropwise over two hours. The resulting slurry was aged at room temperature overnight and isolated by filtration, and the filter cake was washed with 8.3% water/25.0% methanol/66.7% ethanol (4× volumes, 100 mL) and ethanol (4× volumes, 100 mL). The filter cake was dried in a vacuum oven at 50° C. under a nitrogen stream for six hours to afford crude suramin 6 (20.8 g, 81.7% yield).

Crude suramin (235.0 g, 0.164 mol, 1.0 equiv) was slurried in 30% ethanol in methanol (3.525 L, 0.05 M). The slurry was heated to 50° C. with stirring for one hour and subsequently cooled to room temperature for one hour. The resulting slurry was filtered through Qualitative 4 filter paper, and the resulting filter cake was washed with 30% ethanol in methanol (940 mL). The filter cake was dried in a vacuum oven at 40° C. under a nitrogen stream for four hours and then at 60° C. for two days under a nitrogen stream to afford suramin 6 (175.0 g, 72.31% yield, 97.10% purity).

1.-45. (canceled)

46. A pharmaceutical composition comprising a substantially pure composition of a compound of Formula I: 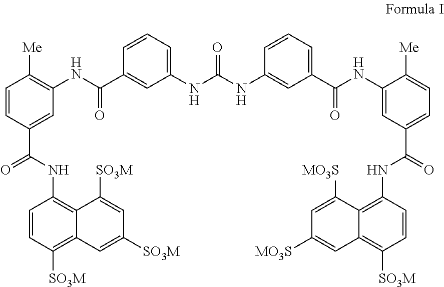
and a pharmaceutically acceptable excipient, wherein M is each independently H, Li, Na, or K.

47. The pharmaceutical composition of claim 46, wherein each M is Li, Na, or K.

48. The pharmaceutical composition of claim 47, wherein each M is Na.

49. The pharmaceutical composition of claim 46, wherein the substantially pure composition of the compound of Formula I comprises, by weight or by mole, at least 95% of the compound of Formula I.

50. The pharmaceutical composition of claim 49, wherein the substantially pure composition of the compound of Formula I comprises, by weight or by mole, at least 97% of the compound of Formula I.

51. The pharmaceutical composition of claim 46, wherein the substantially pure composition of the compound of Formula I comprises, by weight or by mole, about 95% to about 99.9% of the compound of Formula I.

52. The pharmaceutical composition of claim 46, wherein the substantially pure composition of the compound of Formula I comprises an impurity of Formula I-A:

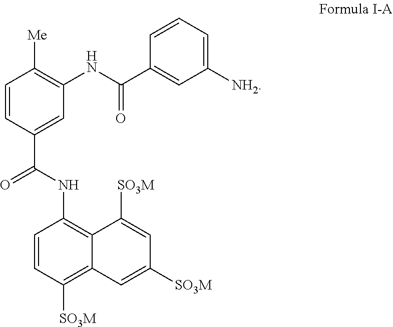

53. The pharmaceutical composition of claim 52, wherein the substantially pure composition of the compound of Formula I comprises, by weight or by mole, less than 5% of the impurity of Formula I-A.

54. The pharmaceutical composition of claim 53, wherein the substantially pure composition of the compound of Formula I comprises, by weight or by mole, less than 3% of the impurity of Formula I-A.

55. The pharmaceutical composition of claim 52, wherein the substantially pure composition of the compound of Formula I comprises, by weight or by mole, about 0.01% to about 5% of the impurity of Formula I-A.

56. The pharmaceutical composition of claim 46, wherein the pharmaceutically acceptable excipient is a solvent.

57. The pharmaceutical composition of claim 46, wherein the pharmaceutically acceptable excipient is saline.

58. A method of treating a disease or disorder in a subject in need thereof, wherein the method comprises administering to the subject a therapeutically effective amount of the pharmaceutical composition of any one of claims 46 to 57, further wherein the disease or disorder is an autism spectrum disorder or fragile X-associated tremor/ataxia (FXTAS).

59. The method of claim 58, wherein the disease or disorder is an autism spectrum disorder.

60. The method of claim 58, wherein the disease or disorder is fragile X-associated tremor/ataxia (FXTAS).

61. The method of claim 58, wherein the pharmaceutical composition is administered to the subject intravenously, subcutaneously, or parenterally.

62. The method of claim 61, wherein the pharmaceutical composition is administered to the subject intravenously.

63. A method of preparing a compound of Formula I-A: 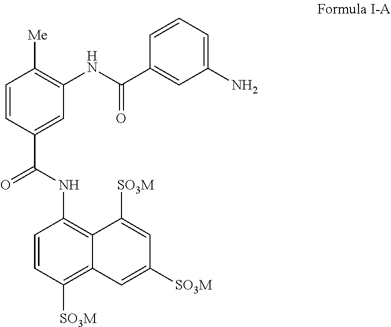
from a compound of Formula I-B: 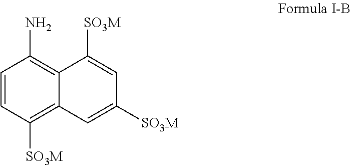
wherein M is each independently H, Li, Na, or K, and wherein the method provides the compound of Formula I-A in an overall yield of greater than 80%.

64. The method of claim 63, wherein the method provides the compound of Formula I-A in an overall yield of greater than 90%.

65. The method of claim 63, wherein the compound of Formula I-A: 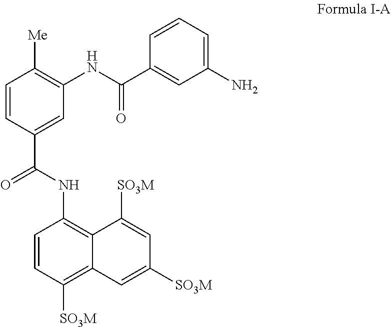
is prepared from the compound of Formula I-B: 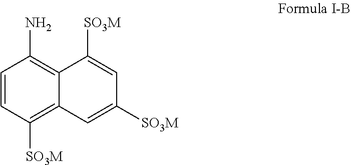
in four synthetic steps.
